(19) United States
(12) Patent Application Publication
(10) Pub. No.: US 2026/0273272 A1
(43) Pub. Date: Sep. 17, 2026

(54) Electrode Lead and Method for Pain Block using Ionic Direct Current

(71) Applicants: THE JOHNS HOPKINS UNIVERSITY, Baltimore, MD (US); Newsouth Innovations PTY Limited, Sydney, New South Wales (AU)

(72) Inventors: Yun Guan, Spark Glencoe, MD (US); Paul Adkisson, Novato, CA (US); Gene Yevgeny Fridman, Baltimore, MD (US); Chaojun Cheng, Baltimore, MD (US); Felix Aplin, Sydney (AU); Gila Moalem-Taylor, Sydney, New South Wales (AU); Mohit Shivdasani, Sydney, New South Wales (AU); Tom Su, Sydney, New South Wales (AU); Yiru Guo, Sydney, New South Wales (AU)

(21) Appl. No.: 19/471,273

(22) PCT Filed: Apr. 1, 2024
(86) PCT No.: PCT/US2024/022499

§ 371 Date: Sep. 30, 2025

Related U.S. Application Data
(60) Provisional application No. 63/493,504, filed on Mar. 31, 2023.

(51) Int. Cl. A61N 1/05; A61N 1/36

(52) U.S. Cl. CPC A61N 1/0556; A61N 1/36017; A61N 1/36021; A61N 1/36034

(57) ABSTRACT

Electrical neuromodulation uses electric current to interfere with nociceptive (pain-related) signals. Delivery of novel ionic direct current (iDC) to peripheral nerves can block nociceptive signal conduction without interfering with non-painful tactile or proprioceptive signals. A cuff lead for delivering iDC is disclosed for blocking action potential (AP) propagation in a neuron. The cuff lead delivers iDC to a nerve via a microfluidic lead filled with conductive media such as a gel. The cuff lead can take the form of a tripolar nerve cuff configured to contact a surface of a peripheral nerve. The cuff lead delivering iDC to the nerve according to the present invention has been shown to block AP propagation in a rat sciatic nerve and can be used as a treatment for blocking pathological pain.

### CROSS REFERENCE TO RELATED APPLICATIONS

This application claims the benefit of U.S. Provisional Patent Application No. 63/493,504 filed on, Mar. 31, 2023, which is incorporated by reference, herein, in its entirety.

### GOVERNMENT RIGHTS

This invention was made with government support under grant NS092726 awarded by the National Institutes of Health. The government has certain rights in the invention.

### FIELD OF THE INVENTION

The present invention relates generally to medical devices. More particularly, the present invention relates to a system and method for pain blocking using energy delivered to a microfluidic cuff which may be in the form of ionic direct current.

### BACKGROUND OF THE INVENTION

Pain management remains a problem for which new solutions are needed and especially in the case of chronic pain. Neuropathic pain is caused by damage to the somatosensory nervous system, and chronic inflammatory pain stems from an extended immune response following tissue injury. Hallmarks of chronic pain include pain hypersensitivity symptoms, such as allodynia (pain in response to non-painful stimuli) and hyperalgesia (exaggerated pain responses).

Treatment of chronic neuropathic and inflammatory pain is difficult. Medications effective against acute pain are not suitable for the prolonged consumption necessary to treat chronic pain due to risks of toxicity, addiction, and abuse. In particular, over-use of opioids has contributed to an addiction epidemic. Anti-epileptic medication (e.g., gabapentin) and tri-cyclic antidepressants provide clinically significant pain relief (>50% reduction) for around one third of neuropathic pain patients, but are accompanied by drowsiness, dizziness, gait disturbance, and peripheral oedema, and prediction of efficacy is difficult. Corticosteroid injections can temporarily relieve intractable inflammatory pain, but there are restrictions on how many can be administered due to accumulating adverse effects. At present, the available conventional medications cannot be considered a satisfactory solution.

Electrical neuromodulation has emerged as an innovative alternative. Delivery of electric current to neural tissue can disrupt nociceptive signals before they reach the brain and are perceived as pain. The earliest example of neuromodulation for pain is the spinal cord stimulator (SCS). Its analgesic mechanism is based on the ‘Gate Theory’ that nociceptive (Aδ/C) signals are attenuated or ‘gated’ in the spinal cord by innocuous tactile (Aβ) signals. Therefore, SCSs traditionally deliver a low-frequency waveform of current to excite dorsal column Aβ fibers, in turn reducing nociceptive signals. Randomized control trials have shown SCS to provide clinically significant relief for ~45% of people. However, orthodromic Aβ excitation causes paraesthesia which many patients find uncomfortable; the effectiveness of current delivery (and thus analgesia) is prone to postural changes; spinal surgery is required for device implantation; and many studies report poor efficacy beyond 12 months.

Novel SCS waveform paradigms (e.g., burst, high frequency) and feedback-loop innovations have mitigated paraesthesia and inconsistent analgesic coverage. While they demonstrate superior efficacy to traditional SCS, >24-month trials of these latest upgrades are yet to be performed. Over 40% of patients still report <80% pain relief. There remains a need to develop treatments for this significant subset of patients whose pain is not mitigated by existing medicines or technologies.

Accordingly, there is a need in the art for an improved method and system for blocking pain.

### SUMMARY OF THE INVENTION

The foregoing needs are met, to a great extent, by the present invention which provides a device including a cuff configured to contact a peripheral nerve. The device also includes microfluidic channels. The microfluidic channels define an inner space. The microfluidic channels are configured to conduct energy such as ionic direct current to the peripheral nerve to selectively suppress the spiking activity of pain transmitting nerve fibers.

In accordance with an aspect of the present invention, the device further includes a conduit of energy. The cuff is formed from a biocompatible material such as silicone. The cuff is configured to maintain contact with a surface of the peripheral nerve. The cuff is coupled to the microfluidic channels, and the cuff contacts the peripheral nerve. Each of the microfluidic channels is filled with a conductive media such as a gel. Each of the microfluidic channels is connected to a direct current source. The microfluidic channels have a tripolar configuration. The tripolar configuration includes a central channel flanked by two return channels. Delivery of ionic direct current occurs in three phases: a 10 s ramp on, 90 s constant ionic direct current delivery, and a 10 s ramp off.

In accordance with another aspect of the present invention, a device includes three channels, wherein each of the three channels defines an inner space. The three channels are configured to conduct energy such as ionic direct current to a target.

In accordance with another aspect of the present invention, the device includes a cuff for delivering energy to the target. The device further includes a conduit of energy. The cuff is formed from a biocompatible material such as silicone. The cuff is configured to contact a surface of a peripheral nerve. Each of the three channels is filled with a conductive media such as a gel. Each of the three channels is connected to a direct current source. The three channels have a tripolar configuration. The tripolar configuration includes a central channel flanked by two return channels. Delivery of ionic direct current occurs in three phases: a 10 s ramp on, 90 s constant ionic direct current delivery, and a 10 s ramp off.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a top-down view of a tripolar lead for delivering ionic direct current (iDC) according to an embodiment of the present invention.

FIGS. 2A-2F illustrate perspective views of a fabrication method of a tripolar lead according to an embodiment of the present invention.

FIG. 3 illustrates placement of a filled tripolar cuff lead and an unfilled tripolar cuff lead placed around the sciatic nerve in the rat.

FIGS. 4A-4D illustrate graphical views of examples of recorded eCAP signal before, during and after the application of iDC delivered via the d=1.4 mm and g=2 mm cuff lead.

FIG. 5 illustrates a graphical view of measured eCAP amplitude (peak to peak) in accordance with the amplitude of applied iDC.

FIG. 6 illustrates graphical views of iDC applied to the rat sciatic nerve attenuates activity of small-diameter (Aδ/C) fibers at lower amplitudes than large-diameter (Aβ) fibers.

FIGS. 7A and 7B illustrate image and graphical views associated with an iDC delivery protocol.

FIGS. 8A and 8B illustrate graphical views of development of pain hypersensitivity in CFA-injected and nerve-injured rats.

FIGS. 9A and 9B illustrate schematic and image views of MEA insertion and recording locations.

FIG. 10 illustrates image views of a noxious thermal physiological stimulus. Noxious thermal stimulation—one of four physiological stimuli paradigms used—was evoked by a 980 nm diode laser pulse (1 ms duration).

FIG. 11 illustrates graphical views of examples of multi-unit voltage traces of evoked neuronal activity.

FIG. 12 illustrates graphical views of examples of evoked single-unit responses extracted by spike-sorting.

FIGS. 13A and 13B illustrate graphical views of net neuronal activity in control (non-iDC) recordings by neuron type.

FIG. 14 illustrates qualitative examples of iDC's effects across phases and stimulation types.

FIGS. 15A and 15B illustrate graphical views of an effect of iDC on net evoked neuronal activity.

FIGS. 16A and 16B illustrate graphical views of an effect of iDC polarity and amplitude on neuronal suppression.

FIG. 17 illustrates a graphical view of recovery times following iDC suppression. Recordings of activity were made for up to 30 minutes after iDC cessation.

FIGS. 18A and 18B illustrate graphical views of net activity in control recordings by neuron modality (type) in healthy animals.

FIG. 19 illustrates a graphical view of an effect of iDC on net neuronal activity in healthy animals.

FIG. 20 illustrates a graphical view of an effect of amplitude on net neuronal activity.

FIGS. 21A-21D illustrate a cuff design and method of insertion, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mmd to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

Electrical neuromodulation uses electric current to interfere with nociceptive (pain-related) signals. Delivery of novel ionic direct current (iDC) to peripheral nerves can block nociceptive signal conduction without interfering with non-painful tactile or proprioceptive signals. A cuff lead for delivering iDC is disclosed for blocking action potential (AP) propagation in a neuron. The cuff lead delivers iDC to the nerve via a microfluidic lead filled with conductive media. The cuff lead can take the form of a tripolar nerve cuff with a self-curling silicone membrane to wrap around the nerve. The cuff lead delivering iDC to the nerve according to the present invention has been shown to block AP propagation in dorsal root ganglion neurons and in the spinal cord dorsal horn and can be used as a treatment for blocking nociceptive pain.

More particularly, direct current (DC) can be applied to block AP propagation along the nerve. In animal studies, DC has been shown to block motor fibers in a sciatic nerve. DC has also been shown to selectively block pain specific Aδ and C fibers at lower amplitudes than those required to block somatosensory and muscle fibers. However, prolonged delivery of DC can damage the nerve due to the harmful byproducts produced by the electrochemical reactions at the metal electrodes. Therefore, the application of DC must be limited in duration, strictly below a threshold determined by the charge injection capacity (CIC) of electrode and the amplitude of applied DC, to avoid nerve damage.

Delivery of DC to nerves as ionic flow (iDC) rather than electrical current is a novel neuromodulation paradigm. iDC complies with safety guidelines, because it is rectified (transformed) from charge-balanced AC using novel engineering devices, avoiding toxic by-products entirely. As DC is still being outputted to the nerve, iDC maintains all the desirable properties of traditional electric DC discussed above. Studies in the vestibular and peripheral nervous systems have demonstrated this in vivo. This is significant because it allows, for the first time, DC to be delivered for long durations in the body safely. There are clear implications for such a neuromodulation paradigm in the context of chronic pain treatment.

To increase the safe duration of DC deliver), an electrolyte-filled lead can be used to separate the nerve from being in direct contact with the electrode. A monopolar lead configuration spreads current in the body and can cause undesired muscle twitching. This muscle twitching has also been observed in patients with a neural implant using monopolar configuration. A bipolar configuration can be used to limit the current spread, but neural excitation can still occur due to the returning current flowing in a direction opposite to the blocking current. In contrast, a tripolar configuration is proposed, herein, to create two blocking regions on both sides of the center electrode to confine the current spread to a small region along the nerve and prevent unintended excitation of the nerve.

An exemplary embodiment of the present invention therefore includes a lead with three microfluidic channels that can securely wrap around the sciatic nerve with a self-curling membrane as a cuff to deliver tripolar ionic direct current (iDC). The lead is made of biocompatible material such as silicone and is fabricated with 3D printing and molding techniques. It should be noted that the use of silicone and 3D printed fabrication are included herein by way of example, and any suitable biocompatible material and method of manufacture known to or conceivable to one of skill in the art can also be used. To demonstrate the functionality of a device and method according to the present invention, two types (3 of each) of cuff leads that differed in the distance between channels and cuff diameters were tested on rat sciatic nerves. In an exemplary use of a device and method according to the present invention, the nerve was blocked for 1 hour via this interface was able to recover with no observed damage.

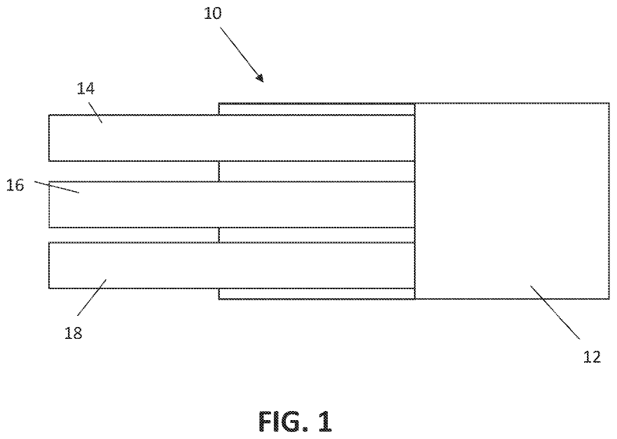

FIG. 1 illustrates a top-down view of a tripolar lead for delivering ionic direct current (iDC) according to an embodiment of the present invention. The tripolar iDC lead 10 includes a self-curling silicone membrane 12 for wrapping around the nerve, and three channels 14, 16, and 18. The channels 14, 16, and 18 are filled with a conductive media such as a gel. The channels 14, 16, and 18 conduct iDC from a direct current source to the nerve. The polarity of the three channels 14, 16, and 18 can be controlled by switching the leads to which it is connected at the source of direct current.

In some embodiments the tripolar lead can take the form of a central channel flanked by two return channels (i.e., tripolar configuration). The polarity of the central channel can be reversed, such that anodic (positive center, negative return) and cathodic (negative center, positive returns) iDC can be delivered. In the anodic configuration. iDC is delivered to the nerve through the cuff's central channel. The two flanking channels act as an isolated electrical return. This tripolar configuration is used because it reduces the current density of the return electrodes and primary electrode “side lobes”, which can induce excitation.

The tripolar lead is configured to be coupled to a source of energy such as direct current in order to deliver the iDC to the nerve. In some embodiments the source of energy can take the form of a freeform stimulator which can deliver ionic direct current without adverse reactions at the electrode interfaces. This decreases potentially toxic electrochemical reactions and increases the duration that the device can be used for treatment.

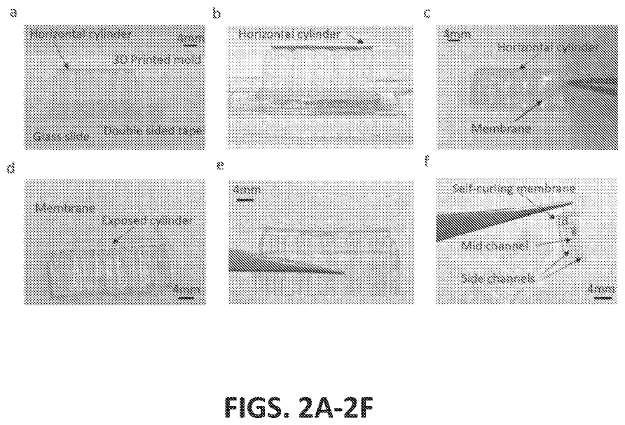

FIGS. 2A-2F illustrate perspective views of a fabrication method of a tripolar lead according to an embodiment of the present invention. The following describes the fabrication of a tripolar lead, according to an embodiment of the present invention. The following description is included herein by way of example and is not intended to be considered limiting. A device according to the present invention can be manufactured with any materials and any fabrication method known to or conceivable to one of skill in the art. A mold structured as a pillar array supporting a horizontal cylinder was 3D printed (FormLabs 3B printer and BioMed Clear Resin, FormLabs, Somerville, MA, US) and was then spray coated with release agent (Ease release 200, Mann release technologies, Macungie, PA, US). In FIG. 2A, the mold was secured onto a glass slide with double sided tape (Nano tape 0.5 inch, Trazon, China) such that the mold would be stable in a spin coating machine. Biocompatible silicone MED2-4220 (Avantor, Radnor Township, PA, US) was selected to fabricate the cuff. The silicone kit contains two liquid parts (A and B) which need to be mixed to initiate curing. First, part A was poured over to thoroughly cover mold surface. The mold was then spun at 1000 RPM (revolutions per minute) for 20 sec to make a thin layer of part A on mold surface. After that, part B was poured over the mold and was then spun under 1000 RPM for 20 sec. The coated mold was then transferred into an oven and cured under 70° C. for 30 minutes. After the first layer of silicone was cured, a second layer of silicone was coated by repeating the previous spin coatings of part A and B and was then cured in oven under 70° C. for 1 hour. The mold coated with cured silicone rubber, illustrated in FIG. 2B, was then removed from the glass slide.

A piece of silicone membrane was attached to the cuff such that the attached membrane can be grasped by tweezer to unfold the cuff. To make the membrane, a glass slide was spray coated with release agent (Ease release 200, Mann release technologies, Macungie, PA, US) and was then spin coated sequentially with part A and B liquid silicone with same spinning rate of 700 RPM for 20 sec for each silicone part. The coated glass slide was then cured under 70° C. for one hour in an oven. A piece of rectangular membrane was cut from the glass slide and was placed on the cuff body with the long edge of membrane aligned with the line right below the horizontal cylinder shown as in FIG. 2C. A small amount of part A and B silicone mixture was applied along the aligning line such that, after being cured, the membrane was firmly attached to the cuff. The mold with the aligned membrane was then transferred into oven for curing (70° C. for 30 minutes).

After that, the attached membrane was flipped over to expose the silicone on the mold surface. A cut was made on the silicone on mold surface along the red dashed line to open the silicone layer on the horizontal cylinder shown, as illustrated in FIG. 2D. Once the silicone layer was opened, the mold was pulled out of silicone, as illustrated in FIG. 2E. The left silicone body was trimmed into five identical cuffs with three channels, as illustrated in FIG. 2F. Each cuff contains three fluid channels (two side channels and one mid channel) and a self-curing membrane which can be wrapped around nerve. The diameter of the self-curing and the distance between the channels were denoted as ‘d’ and ‘g’ respectively in FIG. 2F and can be changed as needed in the mold design.

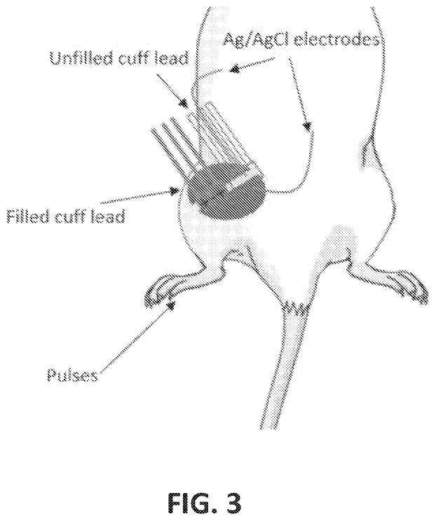

In an exemplary experiment using a tripolar cuff, according to the present invention, electrical pulses (monophasic, amplitude ranged from 2 mA to 5 mA, pulse duration=100 μs, pulse period=0.4 s) were applied to stimulate at a paw of a rat, as illustrated in FIG. 3, with an isolated pulse generator (Model 2100, AM-system, Sequim, WA). FIG. 3 illustrates placement of a filled tripolar cuff lead and an unfilled tripolar cuff lead placed around a sciatic nerve in the rat. The filled cuff lead wraps around the mid-sciatic nerve to deliver iDC with the signs indicating the polarity of each channel. The unfilled lead and the Ag/AgCl electrodes (grey wires) were used to measure the evoked compound action potential (eCAP) induced by the pulses. To increase the signal to noise ratio in eCAP and secure the position of Ag/AgCl electrode, an unfilled cuff lead was wrapped around the nerve with Ag/AgCl wire electrode inserted into the middle channel to contact sciatic nerve. To complete the measurement circuit, another Ag/AgCl wire electrode was placed next to the unfilled cuff lead to touch the surrounding tissue. The measured eCAP signal was processed by a differential AC amplifier (Model 1700, AM-system, Sequim, WA) with a setting of low cut-off=1 Hz, high cut-off=10 kHz, gain=1000, notch=‘ON’. The processed signal was then recorded with CED Power 1401 (Cambridge Electronic Design Limited, Milton, CB, UK). Three micropipette tips (10 μL) were inserted into the three channels of another cuff lead (the filled cuff, illustrated in FIG. 3) to create three electrolyte reservoirs to separately house three stainless steel electrodes (coiled stainless steel wire) coated with PEDOT:PSS (3-4% in H2O, Sigma-Aldrich, St. Louis, MO, US). The cuff lead and reservoirs were filled with Krebs-Henseleit solution (EcoCyte Bioscience, TX, US) to deliver iDC provided by an analog stimulus isolator (Model 2200, AM-system, Sequim, WA). The stainless-steel electrodes placed in the side reservoirs were used as positive electrodes (anodes) which hyperpolarize transmembrane potential. While the electrode in the middle reservoir was used as negative electrode (cathode) which depolarizes the transmembrane potential.

To stimulate all neurons in the sciatic nerve the amplitude of pulses was increased by 0.5 mA increment until reaching the maximum amplitude of eCAP. The pulse amplitude varied due to the wetting condition of paw skin. To search for the minimum iDC required for neural blocking, the current delivered by the filled cuff lead ramped linearly from 0 to 2 mA for 8 seconds, holding at 2 mA for 1 second, then linearly ramped down to 0 mA for another 8 seconds. Because the efficacy of iDC blocking can be impacted by the diameter of self-curling (d) and the distance between the channels (g) due to current spreading and shunting respectively, cuff lead with d=1.4 mm and g=2 mm and cuff lead with d=2 mm and g=1.5 mm were tested within same rat. The latter one was respected to be less effective in neural blocking due to the increased current shunting between the channels and increased current spreading with a loose wrapping. To verify the measured eCAP signal with a negative control, at the end of each rat experiment sciatic nerve was cut at the distal side of the filled cuff lead and was expected to disappear completely.

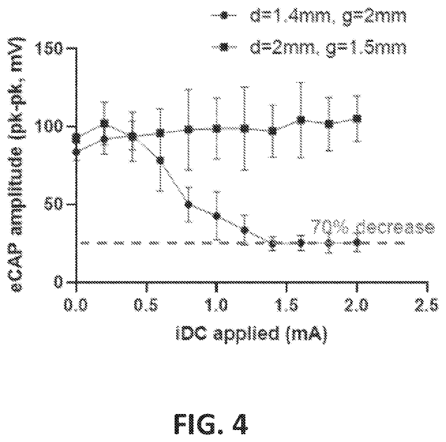

FIGS. 4A-4D illustrate graphical views of examples of recorded eCAP signal before, during and after the application of iDC delivered via the d=1.4 mm and g=2 mm cuff lead. Before the application of iDC, as illustrated in FIG. 4A, the eCAP signal was captured ~2 ms after the artifact induced by the pulse stimulus at paw. The highest and lowest peaks of the lumped eCAP signal were measured and used their difference to be the indicator of neural blocking. To eliminate the impact of random noise, the calculated eCAP difference was deducted with the bound of random noise ranging from 20 mV to 30 mV. FIG. 4B illustrates the eCAP during the application of 2 mA iDC. The amplitude of lumped eCAP signal was obviously reduced indicating an effective neural blocking. FIG. 4C illustrates the recovery of eCAP signal after the iDC was turned off (0 mA). After testing both cuff leads with different ‘d’ and ‘g’, the sciatic nerve was cut at the distal side of the filled cuff lead to verify the eCAP signal. As shown in FIG. 4D (negative control), there was no eCAP signal observed after the sciatic nerve was cut.

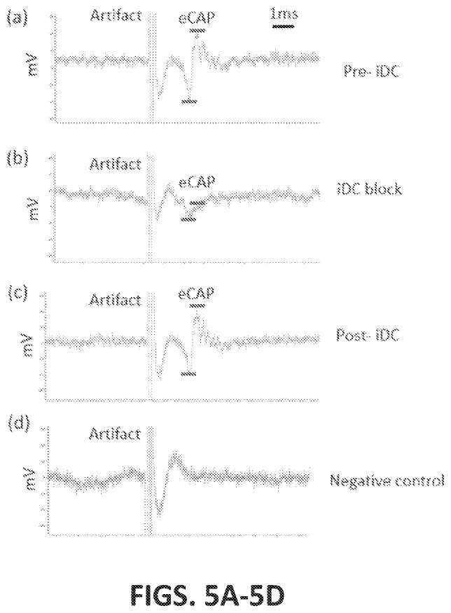

Three cuff leads each with d=1.4 mm/g=2 mm, vs d=1.6 mm/g=1.5 mm (6 cuffs total) were tested for ability to block nerve propagation in three rats. In the (d=1.4 mm/g=2 mm) case (black circles, as illustrated in FIG. 5), the eCAP signal was 83.8±4.4 mV (pk-pk)) when iDC=0 mA and started to decrease after iDC=~0.4 mA. As the applied iDC kept increasing, the eCAP continuously decreased until saturated at its pk-pk amplitude of 27.5±6 2 mA (equivalent to ~70% decrease in eCAP) with the applied iDC=1.4 mA.

FIG. 5 illustrates a graphical view of measured eCAP amplitude (peak to peak) in accordance with the amplitude of applied iDC. The circle plot is the result of the cases tested with the cuff lead with d=1.4 mm and g=2 mm. The square plot is the result of the cases tested with the cuff lead with d=2 mm and g=1.5 mm. The red dashed line represents a 70% decrease in the amplitude of eCAP signal. N=3, three rats were tested with both types of cuff leads, 6 cuff leads were tested in total. Further increase in iDC was not effective in decreasing the eCAP signal. iDC delivered from this cuff could not completely block all neurons in the nerve either due to its geometry or non-ideal electrical isolation. The eCAP signal remained relatively unchanged throughout the application of iDC in experiments conducted with the d=1.6 mm/g=1.5 mm cuffs (bigger diameter, smaller channel distance), as illustrated in FIG. 5. iDC clearly had no impact on the nerve block using this cuff. This is due to shunting between the channels around the nerve. The larger diameter and closer distance between the channels forced the current to short circuit, preventing the current from entering the nerve.

A cuff lead that can deliver iDC to block action potential propagation in the sciatic nerve. The threshold current using tripolar configuration for an effective neural block was tested on rats by ramping the amplitude of the applied iDC and measuring the eCAPs in the sciatic nerve. The required amplitude of current for an effective neural block was ~1.4 mA with d=1.4 mm and g=2 mm cuff lead. In contrast, the cuff leads with d=2 mm and g=1.5 mm were ineffective in blocking the AP conduction with the same iDC range from 0 mA to 2 mA. The difference in the results obtained with the different cuff leads implies that the geometric parameters (the diameter of the curl and the distance between the channels) may be important factors influencing the threshold of iDC for neural block. To optimize the cuff lead and minimize the threshold current, more values of the geometric parameters can be tested.

In another exemplary implementation of the device and method of the present invention, that is not meant to be considered limiting, two distinct animal models were used to test iDC's effects in chronic neuropathic and inflammatory pain conditions. Behavioral tests confirmed that injured animals developed pain hypersensitivity. In vivo electrophysiological recordings from the spinal cord before, during, and after iDC delivery to the sciatic nerve revealed iDC's selective effects on neuronal activity in chronic pain conditions. Specifically, nociceptive-evoked neuronal activity was significantly suppressed at 500 μA and 1000 μA, while innocuous-evoked activity remained unaffected in both inflammatory and neuropathic pain models. There were no differences in effects between different iDC amplitudes or between positive and negative iDC. Of the neurons that recovered following iDC cessation, those evoked by nociceptive stimuli took significantly longer to recover than innocuous-evoked neurons. Together, the results strongly support iDC, and the device and method of the present invention, as a novel treatment for inflammatory and neuropathic pain, particularly medication-resistant conditions affecting the periphery.

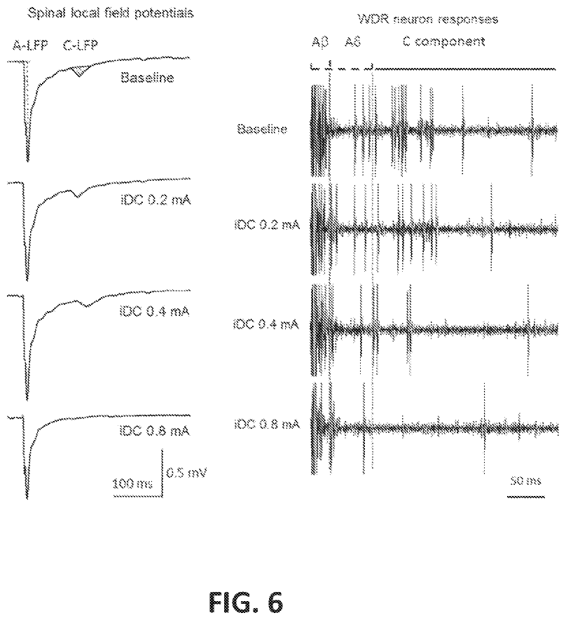

iDC blocks small-diameter nerve fibers at lower amplitudes than large-diameter fibers, as illustrated in FIG. 6. FIG. 6 illustrates graphical views of iDC applied to the rat sciatic nerve attenuates activity of small-diameter (Aδ/C) fibers at lower amplitudes than large-diameter (Aβ) fibers. At low amplitudes (0.2-0.4 mA) the C-LFP (i.e., the contribution of C-fibers to the local field potential, left) and Aδ/C components of WDR neuronal activity (right) are partially reduced compared to baseline, while the A-LFP (left) and Aβ WDR components (right) are unaffected. At the highest amplitude (0.8 mA) the C-LFP and Aδ/C components become fully attenuated while the A-LFP and Aβ components are only partially reduced. This novel phenomenon contradicts the long-held axiom that nerve block thresholds are inversely proportional to fiber diameter.

It is known that VGSCs are differentially expressed on nerve fibers of different diameters; These differences in channel expression dictate block threshold, rather than fiber diameter size per se, to explain their results. This is significant as small-diameter (Aδ/C) fibers conduct nociceptive signals while large-diameter (Aα/Aβ) fibers conduct innocuous light touch and proprioceptive signals. iDC is therefore capable of achieving nociceptive-specific nerve block. Pain relief without disruption of other sensory modalities like touch or proprioception would be a significant step forward in pain treatment. No other device or waveform paradigm has achieved this.

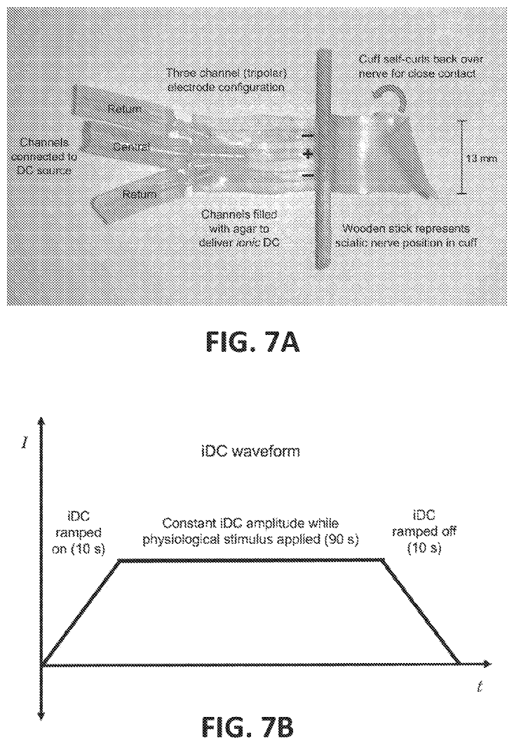

FIGS. 7A and 7B illustrate image and graphical views associated with an iDC delivery protocol. FIG. 7A illustrates an image view of a silicone nerve cuff electrode used to deliver iDC. It consists of a central channel flanked by two return channels (i.e., tripolar configuration). The polarity of the central channel could be reversed by switching the leads coming from the A-M Systems DC source (not pictured). This enabled testing of anodic (positive center, negative return) and cathodic (negative center, positive returns) iDC. Pictured is the anodic configuration. FIG. 7B illustrates a graph of the specific iDC waveform used in this study. Ramping (10 s on/off) mitigated onset and offset excitation. Constant phase amplitudes of 500 and 1000 μA were tested. The constant iDC duration was 90 s for all trials.

In the exemplary implementation of FIGS. 7A and 7B, a tripolar silicone nerve cuff electrode, according to the present invention, was fitted around the left sciatic nerve, proximal to the site of injury. This involved surgically exposing the sciatic nerve and clearing away connective tissue to allow sufficient space. The cuff electrode used included a central channel flanked by two return channels, as illustrated in FIG. 7A. These channels were individually filled with electrolytic agar gel and connected to an A-M Systems 4100 Isolated High-Power Stimulator (Sequim, USA) that served as a DC power source. DC was thus delivered to the nerve via a stream of ions, or ionic direct current (iDC), rather than via a metal electrode. iDC was delivered to the nerve through the cuff's central channel. The two flanking channels acted as the isolated electrical return. This tripolar configuration was used, because it reduces the current density of the return electrodes and primary electrode “side lobes”, which can induce excitation.

iDC delivery occurred in three phases: 10 s ramp on, 90 s constant iDC delivery, 10 s ramp off, as illustrated in FIG. 7B. Ramping mitigates onset and offset responses. Constant phase iDC amplitudes of 500 and 1000 μA were tested based on previous studies and known block thresholds of sensory fibers. A recovery period lasting the duration of the steady phase was given between each iDC application. The polarity of the channels was reversed to test both anodic (positive) and cathodic (negative) iDC.

Spinal neuronal responses were evoked by physiologically stimulating the skin of the injured hindpaw. Four different stimulation paradigms were used: two noxious (painful) and two innocuous (non-painful). This allowed nociceptive and non-nociceptive spinal responses to be evoked, respectively. All stimuli were applied to the plantar surface of the hindpaw except the proprioceptive stimulus, which rotated the ankle and knee joints.

Noxious thermal spinal responses were evoked by heating the paw with a 980 nm diode laser (Changchun Optoelectronics Technology Co., Changchun, PRC). Beam intensities across all experiments ranged 3.75-14.6 W depending on stimulation location (e.g., paw pad vs. digit) and individual animal threshold differences. Beam diameter remained constant (4 mm). One full set of recordings consisted of 3 single repetitions (pulses). Pulses lasted 1 ms and were spaced ~100 s apart. A ThermaCAM Reporter7 Pro thermal camera (FLIR Systems, Oregon, USA) was used to confirm the paw surface temperature upon laser stimulation. Image analysis was performed in ImageJ.

Noxious pinch responses were evoked by firm, manual presses of the paw with a wooden rod. A pressure sensor under the paw detected onset timings of each press. One full set of recordings consisted of 5 sets of 10 repetitions (presses). Presses lasted ~0.25 s and were made at ~1 Hz.

Tactile responses were evoked by a SignalForce V4.0 shaker (DataPhysics, Santa Clara, USA) connected to a lightweight aluminum rod (1 mm diameter). The shaker was programmed to displace by 100 μm which gently pressed the rod into the paw, giving it a light touch. One full set of recordings consisted of 5 sets of 10 repetitions (presses) delivered at 1 Hz.

Proprioceptive responses were evoked by a wooden rod attached to a stepper motor (Pololu Corp., Las Vegas, USA) positioned under the ankle. The motor was programmed to rotate 15° over 200 ms and then return to starting position, per repetition. This briefly rotated the ankle and knee joints. One full set of recordings consisted of 5 sets of 10 repetitions (rotations) delivered at 1 Hz.

Recordings of evoked spinal activity in response to stimulations of the injured hindpaw were made before, during, and after iDC delivery. Pre-iDC (control) recordings were of neuronal activity evoked by the physiological stimulus alone. During-iDC (test) recordings were of activity evoked by the physiological stimulus while iDC was delivered to the sciatic nerve. Post-iDC (recovery) recordings were of evoked activity following iDC cessation. iDC block has been reported to persist after iDC delivery has stopped; therefore, recovery recordings were made at intervals of 1-5 minutes for up to 30 minutes following iDC cessation, or until the evoked response qualitatively returned. Digital triggers aligning with the onset of physiological stimulations informed recording timings. Spontaneous activity (i.e., without physiological stimuli or iDC) recordings were made to ensure digital triggering of the stimulation equipment was not itself evoking neuronal activity. 3-5 trial sets were collected for each condition (3 for laser trials, 5 for other stimuli). All recordings were made using the 32-channel MEA connected to an INTAN RHS recording and data acquisition system (Intan Technologies, LA, USA). This system recorded voltage changes across each recording channel of the MEA at a sampling rate of 30 kHz.

Processing of electrophysiology data was done using custom programming scripts written in the high-level language, Julia. Raw MEA recordings were first sorted into trials based off digital trigger timing information. All recording signals were band-pass filtered (300-5000 Hz) then visualized in the code-editing software. Visual Studio Cde (Microsoft, WA, USA). Multi-unit responses were selected for further analysis if they were clearly evoked by a physiological stimulus and present across at least three adjacent channels.

For each group of three adjacent channels exhibiting similar responses, spikes occurring at the same time and with identical shapes were concatenated into a single waveform. The shape of this waveform was analyzed by principal components analysis (PCA) and the principal components clustered using an unsupervised k-means algorithm. This created a waveform template representing the typical spike profile of an individual neuron. For example, three similar channel responses evoked by the tactile stimulus would be chosen, and the principal features of each spike would be analyzed via PCA to create templates that represented the spike shapes of unique ‘tactile’ neurons.

This was repeated for various channels and recording locations to generate unique templates for every individual neuron encountered. Templates were then matched against spike trace recordings evoked by other physiological stimuli. Individual neurons were classified as tactile-, proprioceptive-, noxious thermal-, or noxious pinch-dominant based on the type of physiological stimuli they most strongly responded to. To observe the effects of iDC, neuron templates generated from pre-iDC recordings were matched against their during-iDC and post-iDC recordings. Statistical analysis of the differences in the number of evoked spikes before vs. during vs. after iDC allowed quantitative analysis of iDC's effects on neuronal activity.

Statistical analysis was performed in R (R Core Team, 2022) using custom scripts. For each behavioral test, changes to withdrawal thresholds or latencies were analyzed using a 2-way-ANOVA. This considered changes over time (pre-injury vs. day 3 vs. day 4/7) as well as changes between hindpaws (injured vs. uninjured). Where significant effects were found, comparisons were made using Tukey p-values (emmeans function in R). Data are visualized and reported as individual points and estimated marginal means (EMM) with 95% confidence intervals (CI) and degrees of freedom (df). Significance was defined as p<0.05.

The number of single-unit spikes before and after each physiological stimulation trial within equal-duration windows were counted for each recording, using a custom Julia script. Window timings were based off expected peak response times, which varied depending on stimulus type, as shown in Table 1. An ‘evoked’ response was defined as having more spikes in the post-trial window than the pre-trial window (determined by a paired t-test, p<0.05) and with a mean net response of ≥1 spike per trial (calculated by subtracting the pre-stimulus count from the post-stimulus count). Only neurons which displayed an evoked response according to these criteria were further analyzed. Additionally, to control for tactile cells' low activation thresholds which resulted in them being activated by all stimuli, analysis on proprioceptive and nociceptive neurons was restricted to only those that didn't also respond to the tactile stimulus.

Statistical analysis was performed in R using custom scripts. Evoked neuronal responses were examined for changes in the number of evoked spikes before, during, and after iDC. Pre-iDC recordings served as controls to which during- and post-iDC recordings were compared. ANOVA statistics were not appropriate to analyze variance in this population due to variable interdependence. Effects of iDC were instead determined using linear mixed-effects models with Satterthwaite's method to account for random factors and nested data (LMER; lmer function in R). Fixed factors in both models included physiological stimulus, injury model, experimental phase, iDC amplitude, and recovery time. The LMER random factors were unique animal and neuron identifiers. Model fitting was assessed by inspection of residual plots. Where the LMER analysis found significant interactions between factors, post-hoc analysis using Tukey's test determined how this interaction affected neuronal activity. All data are reported as EMMs with 95% CI's and df and visualized as individual points with boxplots. Significance was defined as p<0.05.

TABLE 1
Window timings for various physiological stimuli recordings. Timings
represent the windows used to count and compare number of spikes,
to determine the amount of evoked neuronal activity. Windows are
based off expected peak response times, which vary according to
conduction velocities of different neuron types. Times are relative
to the onset of the physiological stimulus (0 ms). Pre- and post-
windows are of equal duration within each stimulus type.

| Physiological | Pre-stimulus | Post-stimulus |
|---|---|---|
| stimulus | window (ms) | window (ms) |
| Tactile | −50-0 | 0-50 |
| Proprioceptive | −400-0 | 0-400 |
| Noxious pinch | −500-0 | 100-600 |
| Noxious thermal | −800-0 | 1000-1800 |

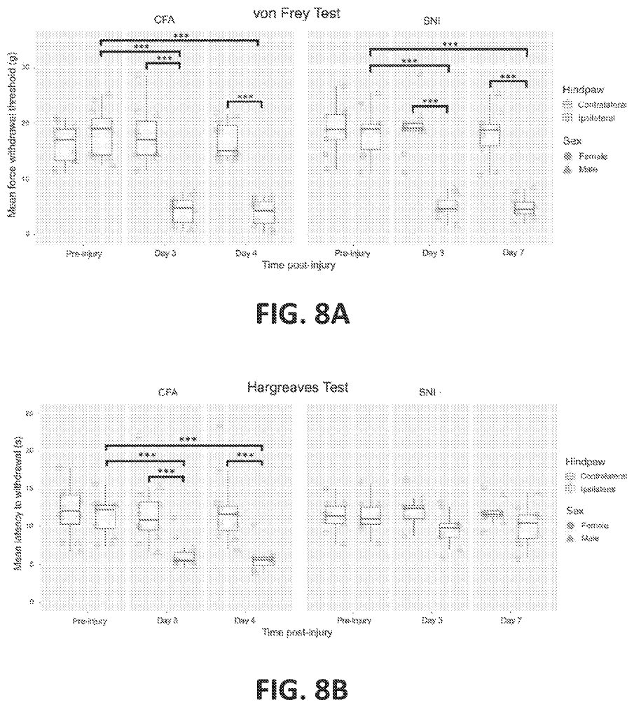

FIGS. 8A and 8B illustrate graphical views of development of pain hypersensitivity in CFA-injected and nerve-injured rats. The von Frey and Hargreaves tests measured mechanical allodynia and thermal hyperalgesia, respectively. Both tests were performed on all rats (CFA n=12; SNI n=10) prior to injury and then on days 3 and 4 (CFA rats) or days 3 and 7 (SNI rats) post-injury. CFA rats had 100 μL CFA injected subcutaneously into the left hindpaw. SNI rats had two out of three (⅔) of their left sciatic nerve branches ligated and axotomized, sparing only the tibial branch. FIG. 8A illustrates the von Frey results. In both animal models, ipsilateral (injured) hindpaw withdrawal thresholds significantly decreased following injury (days 3 and days 4/7) compared to pre-injury (p<0.001), and these thresholds were significantly lower than the contralateral side (p<0.001). FIG. 8B illustrates the Hargreaves results. In the CFA but not SNI model, ipsilateral latencies significantly decreased following injury (days 3 and 4) (p<0.001), and these latencies were significantly lower than the contralateral side (p<0.001).

To confirm the development of allodynia following unilateral injury (CFA injection or SNI of the left hindpaw), mechanical withdrawal thresholds were measured using the von Frey Test. In CFA rats, hindpaw side and time post-injury were found to have a significant effect on withdrawal thresholds (LM, both p<0.001, n=12 rats), as shown in FIG. 8A and interaction with one another (LM, p<0.01). Post-hoc analysis revealed that ipsilateral (injured) hindpaw thresholds were significantly lower on days 3 (EMM=4.28; CI=2.27, 6.29; df=63; Tukey p<0.001) and 4 (EMM=3.83; CI=1.74, 5.93; df=63; Tukey p<0.001) compared to baseline (EMM=18.35; CI=16.34, 21.36; df=63). The ipsilateral threshold did not significantly change from day 3 to day 4 (p=0.95). There were no significant changes in contralateral thresholds (LM, all p>0.58) or any sex effects (LM, p=0.091).

In SNI rats, hindpaw side, time post-injury, and sex each had a significant effect on withdrawal thresholds (LM, p<0.001 for time, p<0.001 for side, p=0.002 for sex, n=10 rats), as illustrated in FIG. 8A. Time and hindpaw side had a significant interaction with one another (LM, p<0.001). Post-hoc analysis showed that ipsilateral but not contralateral withdrawal thresholds were significantly lower on days 3 (EMM=5.06; CI=2.64, 7.48; df=46; Tukey p<0.001) and 7 (EMM=4.90; CI=2.58, 7.23; df=46; Tukey p<0.001) compared to baseline (EMM=18.89; CI=16.57, 21.22; df=46). The mean ipsilateral threshold did not significantly change from days 3 to 7 (LM, p=0.99). Sex had no significant interactions with other factors (all p>0.21), but males had higher withdrawal thresholds across all trials (EMM=15.9; CI=14.4, 17.4; df=46, Tukey p=0.0022) compared to females (EMM=12.8, CI=11.5, 14.0; df=46).

The Hargreaves Test was used to confirm thermal hyperalgesia by measuring latency to withdrawal from a noxious thermal stimulus. In CFA rats, hindpaw side, time post-injury, and sex each had significant effects on withdrawal thresholds (LM, p=0.0048 for time, p<0.001 for side, p=0.025 for sex, n=12 rats), as illustrated in FIG. 8B. Time and hindpaw side had a significant interaction with one another (LM, p=0.0038). Post-hoc analysis revealed that ipsilateral withdrawal thresholds were significantly lower on days 3 (EMM=6.27; CI=4.44, 8.11; df=58; Tukey p<0.001) and 4 (EMM=5.68; CI=3.78, 7.58; df=58; Tukey p<0.001) compared to baseline (EMM=11.36; CI=9.52, 13.20; df=58). The ipsilateral latency did not significantly change from days 3 to 4 (LM, p=0.90). No significant differences in contralateral latencies occurred (LM, all p>0.96). Sex had no significant interactions with other factors (all p>0.18), but females had a higher mean latency across all trials (EMM=10.68; CI=9.69, 11.7; df=46, p=0.024) compared to males (EMM=8.93; CI=7.78, 10.1; df=46).

In SNI rats, hindpaw side was the only factor to have a significant effect on withdrawal latency (LM, p=0.017, n=10 rats). The mean latency for contralateral hindpaws (EMM=11.8; CI=10.98, 12.7; df=46; Tukey p=0.022) was found to be significantly higher on average across all Hargreaves trials compared to ipsilateral hindpaws (EMM=10.4; CI=9.58, 11.3; df=46). No other significant effects were observed (LM, p=0.81 for time, p=0.99 for sex).

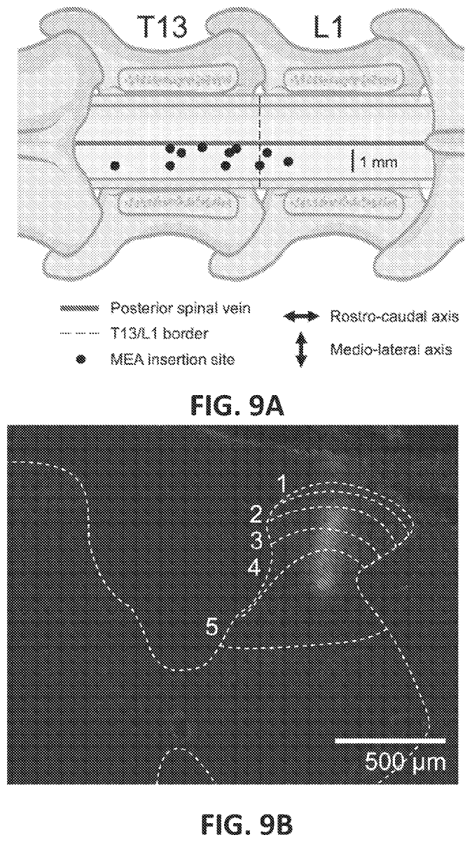

FIGS. 9A and 9B illustrate schematic and image views of MEA insertion and recording locations. FIG. 9A illustrates a schematic showing various locations across the dorsal surface of the spinal cord where the 32-channel recording MEA w as inserted. The intersection of the T13/L1 border and posterior spinal vein was defined as the (0,0) coordinate and locations were measured relative to this using a stereotaxic frame. Measurements are accurate in the rostro-caudal and medio-lateral axes to ±0.1 mm. FIG. 9B illustrates an image view of a transverse section of the spinal cord at the level T13. The white arrow points to an example trace of an MEA tract from one of the experiments Cryosectioning for this image was performed by Catherine Guo. Cross-reference to an anatomical atlas confirmed the location in the dorsal horn laminae I-V.

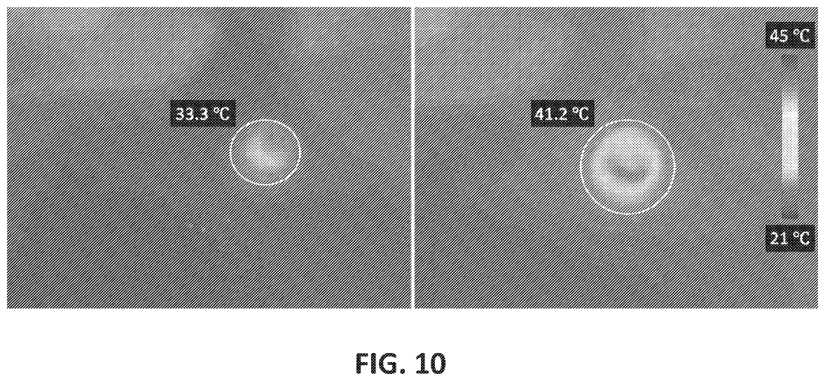

FIG. 10 illustrates image views of a noxious thermal physiological stimulus. Noxious thermal stimulation—one of four physiological stimuli paradigms used—was evoked by a 980 nm diode laser pulse (1 ms duration). The plantar hindpaw surface is shown during laser stimulation at 3.75 (left) and 14.6 (right) Watts. This represents the range used across experiments. The labelled temperature for each spot represents the maximum within the circular outline and is given in degrees Celsius. Images were taken with a ThermaCAM.

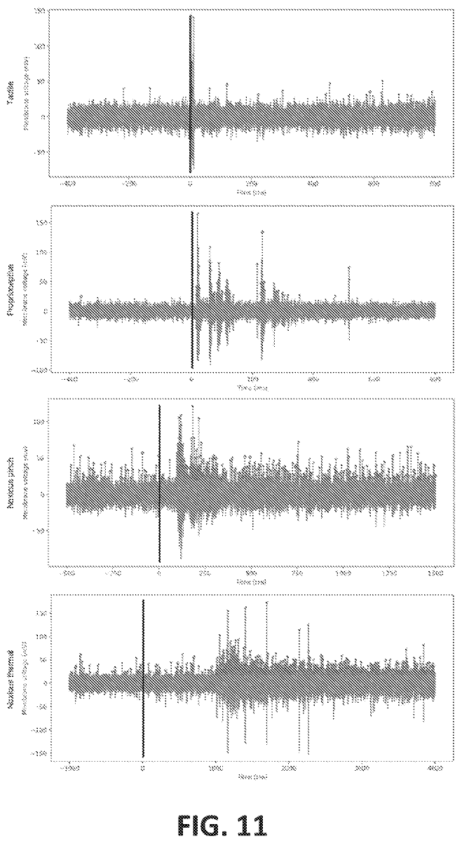

FIG. 11 illustrates graphical views of examples of multi-unit voltage traces of evoked neuronal activity. Traces show the visualization of raw recordings obtained from the MEA as a plot of voltage over time. Each trace shows the voltage changes of multiple neurons (i.e., multi-unit) in response to a single trial of the respective physiological stimulus (tactile, proprioceptive, noxious pinch, noxious thermal). Red dots represent responses identified as being above threshold (i.e., an action potential spike).

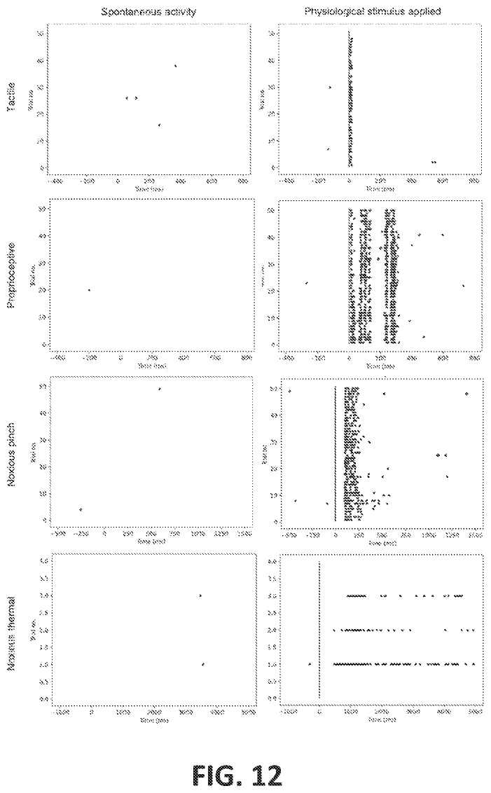

FIG. 12 illustrates graphical views of examples of evoked single-unit responses extracted by spike-sorting. Raster plots show spike activity of a single neuron at rest (i.e., spontaneous activity, left) and then in response to a physiological stimulus (right). Rasters were extracted by spike-sorting protocol and are arranged by neuron classification. For example, the tactile rasters (top) show responses of a neuron whose template was derived from the tactile stimulus, and which responds most strongly to the tactile stimulus, and has thus been classified as a tactile-dominant neuron. Within each raster, each row represents one physiological stimulation trial. For stimulation trials, the vertical bar at 0 ms represents the onset of the stimulus. For each raster shown here, the stimulus applied corresponded to the neuron type (e.g., the tactile stimulus was applied to the tactile-dominant neuron).

Once pain hypersensitivity had been established, in vivo spinal electrophysiology experiments were performed. Neuronal activity was recorded from the dorsal spinal cord at the levels T13-L1, as illustrated in FIG. 9. FIG. 10 shows an example physiological stimulus (noxious thermal) being applied to evoke neuronal activity. Visualization of raw multi-unit voltage traces showed qualitatively that neuronal activity could be captured, and that additional neuronal activity could be evoked by physiologically stimulating the hindpaw, as illustrated in FIG. 11. Generation of raster plots qualitatively confirmed that spike-sorting could extract single-unit activity from these multi-unit traces, as illustrated in FIG. 12.

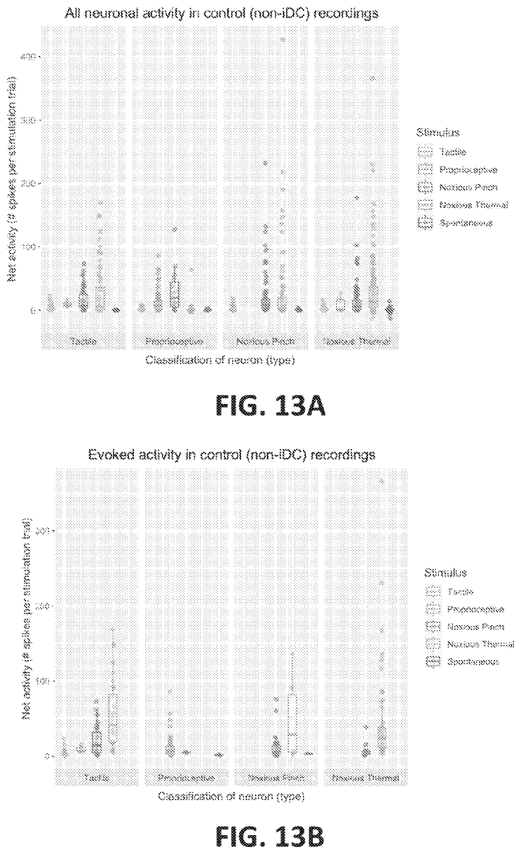

FIGS. 13A and 13B illustrate graphical views of net neuronal activity in control (non-iDC) recordings by neuron type. Recordings from a total of 512 neurons were obtained across all experiments. The number of action potential spikes of each neuron per stimulation trial (i.e., net activity) is plotted against neuron type and arranged by the type of stimulus applied to evoke the response. All recordings are in the absence of iDC. FIG. 13A illustrates net responses from all control (non-iDC) recordings made (n=512). FIG. 13B illustrates net control responses from neurons which passed the criteria for an evoked response (n=220). Only these neurons were further analyzed. Note the absence of activity for the two noxious-classified neurons in response to the innocuous stimuli; these neurons were activated exclusively by noxious stimuli.

Across all electrophysiology experiments (n=22 rats), recordings from a total of 512 unique neurons were obtained (104 tactile, 131 proprioceptive, 179 noxious pinch, 98 noxious thermal). FIG. 13A illustrates the net activity of these neurons in response to physiological stimulation of the injured hindpaw across all control recordings (i.e., recordings in the absence of iDC). 220 of the 512 neurons passed criteria for an evoked response (78 tactile, 53 proprioceptive, 36 noxious pinch, 53 noxious thermal). FIG. 13B illustrates the activity of only these evoked neurons in response to the four physiological stimuli. While some of the evoked tactile and proprioceptive neurons also responded to noxious stimuli, no evoked nociceptive (pinch or thermal) neurons responded to any innocuous stimuli.

After establishing that evoked neuronal activity could successfully be collected and classified, the effects of iDC were examined by recording activity before, during, and after iDC delivery. Data was analyzed across a total of n=195 evoked neurons from 8 CFA (4M/F) and 8 SNI (4M/4F) rats. The impedance of the cuff electrode delivering the iDC ranged from 70-100 kΩ at 1 Hz. This was sufficiently low to allow maximum current (1000 μA) to be delivered without exceeding compliance. iDC recordings were grouped into three experimental phases, pre-iDC, during-iDC, and post-iDC. To examine iDC's effects on different neuron types, recordings were also grouped according to the physiological stimulus that was applied for that recording.

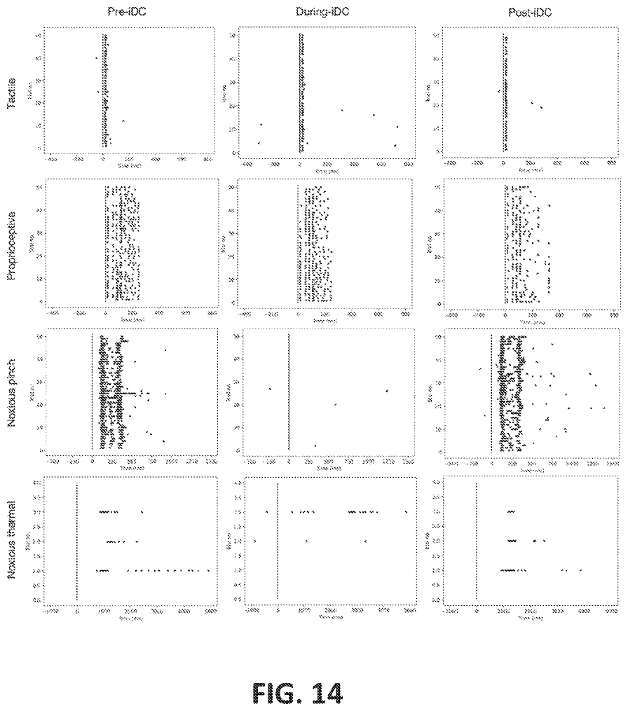

FIG. 14 illustrates qualitative examples of iDC's effects across phases and stimulation types. FIG. 14 illustrates graphical views of examples of single-unit activity before, during, and after iDC. Rasters are arranged according to dominant cell type (i.e., tactile, proprioceptive, noxious pinch, noxious thermal) and experimental phase (i.e., pre-, during-, post-iDC). Within each raster, each row represents one physiological stimulation trial. The vertical bar at 0 ms represents the onset of the stimulus. The during-iDC amplitude in these examples is at 500 μA. Note the pronounced reduction in activity following the noxious pinch and noxious thermal stimuli, but not the tactile or proprioceptive stimuli, during iDC delivery (middle panels).

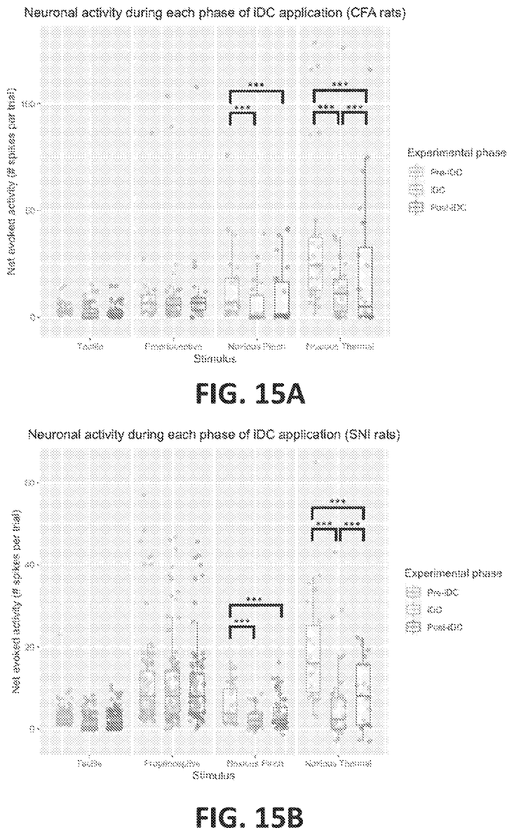

FIGS. 15A and 15B illustrate graphical views of an effect of iDC on net evoked neuronal activity. Spinal recordings of neuronal activity evoked by physiologically stimulating the injured hindpaw were made before, during, and after iDC. Results are arranged according to experimental phase and stimulus type for both FIG. 15A, which shows CFA rats (n=80 neurons/8 rats) and FIG. 15B, which shows SNI rats (n=115 neurons/8 rats). In both injury models, iDC significantly reduced the number of spikes evoked by the noxious pinch and noxious thermal stimuli but not the tactile or proprioceptive stimuli (all p<0.001). Across all trials, evoked activity was significantly greater in CFA rats than SNI rats (note y-axis difference). Note raw data is visualized with medians and interquartile ranges to show its spread, but statistics are reported as estimated marginal means and confidence intervals for pair-wise assessment of iDC's effects on individual neurons. *** p<0.001.

Experimental phase (pre, during, and post-iDC), stimulation type (tactile, noxious pinch, etc.), and injury model (CFA, SNI) were each found to have a significant effect on neuronal activity (LMER, p<0.001 for phase: p<0.001 for stimuli; p=0.022 for injury n=195 neurons/16 rats). There was a significant interaction between experimental phase and stimulation type (LMER, p<0.001) but no other significant interactions (LMER, p>0.05). Post-hoc analysis showed that in response to the noxious thermal stimulus, neuronal activity significantly decreased during-iDC (EMM=12.83; CI=8.53, 17.13; df=237; Tukey p<0.001) compared to pre-iDC (EMM=28; CI=23.70, 32.30; df=239) in both CFA-injected rats, as illustrated in FIG. 15A, and SNI rats, as illustrated in FIG. 15B. When the iDC was turned off, activity significantly increased (post-iDC EMM 16.36; CI=11.91, 20.81; df=271; Tukey p=0.0054) compared to during-iDC; however, activity did not recover back to baseline as there w as still a significant difference between the pre- and post-iDC phases (Tukey p<0.001), as illustrated in FIGS. 15A and 15B. iDC similarly caused a significant reduction in responses to the noxious pinch stimulus (EMM=4.38; CI=−0.27, 9.03; df=220; Tukey p<0.001) compared to baseline (EMM=9.27; CI=44.61, 13.93; df=222). Activity remained significantly suppressed in the post-iDC recovery phase (EMM=6.58; CI=1.97, 11.19; df=213; Tukey p=0.032) and this was not significantly higher than the during-iDC phase (Tukey p=0.089). iDC did not have any significant effects on neuronal activity evoked by the tactile (Tukey p=0.18) or proprioceptive (Tukey p=0.55) stimuli in both CFA-injected rats, as illustrated in FIG. 15A and SNI rats, as illustrated in FIG. 15B. CFA rats displayed significantly greater evoked neuronal activity overall (EMM=11.37; CI=8.59, 14.15; df=201; p=0.016) compared to SNI rats (EMM=6.79; CI=4.32, 9.25; df=188), although activity did not differ across phase or stimulation type by injury model (LMER, all p>0.10). No sex differences were observed (LMER, p=0.13).

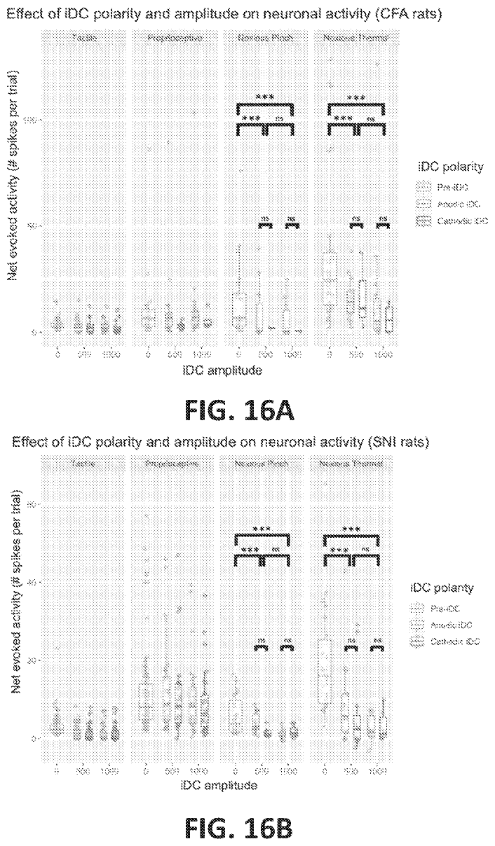

FIGS. 16A and 16B illustrate graphical views of an effect of iDC polarity and amplitude on neuronal suppression. Neuronal activity is grouped by iDC polarity and amplitude. Significant reductions in noxious pinch and thermal-evoked activity were observed during both 500 and 1000 μA iDC; however, there was no significant difference between the effects of 500 vs. 1000 μA for any condition. Similarly, there was no significant difference between anodic vs. cathodic iDC effects for any condition. Effects were the same in both the CFA (top, n=80 neurons/8 rats) and SNI (bottom, n=115 neurons/8 rats) pain models. As above, raw data are visualized with medians and interquartile ranges, but statistics are reported as estimated marginal means and confidence intervals *** p<0.001; ns=not significant.

The amplitude and polarity of current have previously been reported to impact how electrical neuromodulation affects neuronal membrane potentials, especially in computational modelling studies. To investigate if these factors changed how iDC suppressed neuronal activity in chronic pain models in vivo, both anodic (positive) and cathodic (negative) iDC amplitudes of 500 and 1000 μA were tested. The results for CFA and SNI models are presented separately, as illustrated in FIGS. 16A and 16B, because overall activity in these models differed, as shown above.

iDC amplitude was found to have a significant effect on neuronal activity (LMER, p<0.001, n=195) and interaction with stimulation type (LMER, p<0.001). At 500 μA there was significantly less neuronal activity evoked by the noxious pinch (EMM=5.21; CI=0.38,10.05; df=291; Tukey p=0.0091) and noxious thermal (EMM=12.69; CI=8.37, 17.01, df=297, Tukey p<0.001) stimuli compared to 0 μA (pinch EMM=9.87; CI=5.35, 14.40; df=230; thermal EMM=26.99; CI=22.89, 31.10; df=252). This was also true for 1000 μA (pinch EMM=3.24; CI=−1.80, 8.27, df=333; Tukey p<0.001; thermal EMM=9.65; CI=5.21, 14.08; df=324; Tukey p<0.001). However, post-hoc analysis showed that the effects of 500 and 1000 μA were not significantly different from one another for any stimulation type (all Tukey p >0.16). Effects w ere the same in both injury models (LMER, p=0.26), as illustrated in FIGS. 16A and 16B.

Similarly, iDC polarity was reported to have a significant effect on net neuronal activity (LMER, p<0.001, n=195) and have an interaction with stimulation type (LMER, p<0.001); however, post-hoc analysis revealed that all significant differences were between pre-iDC and anodic/cathodic iDC (i.e., between pre- and during-iDC experimental phases). The effects of anodic vs. cathodic iDC were not significantly different from one another at either 500 or 1000 μA across any of the stimulation types (all Tukey p >0.55). Effects were the same in both injury models (LMER, p=0.26), as illustrated in FIGS. 16A and 16B.

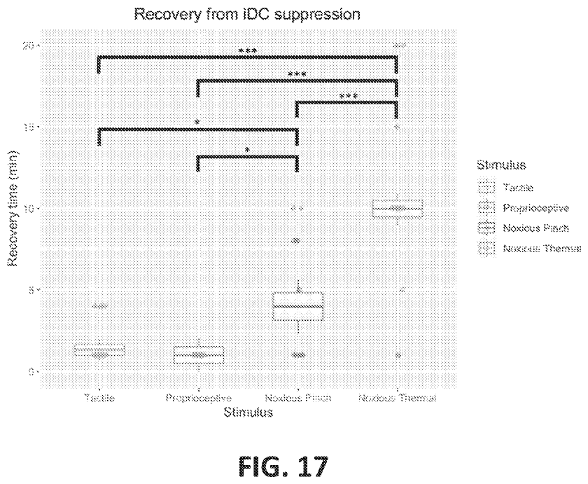

FIG. 17 illustrates a graphical view of recovery times following iDC suppression. Recordings of activity were made for up to 30 minutes after iDC cessation. A neuron was considered fully recovered once its activity was not statistically significantly different from its pre-iDC level (assessed by paired t-test, p<0.05). The figure shows the average time taken for neurons (n=182) to reach full recovery, arranged by stimulation type. Neurons evoked by the noxious thermal stimulus (i.e., nociceptive neurons) took significantly longer than neurons evoked by any other stimulus. Pinch-evoked neurons (i.e., nociceptive neurons) took longer than the two innocuous stimuli. The innocuous-evoked neurons recovered the quickest and did not differ in time to recovery. There were no significant differences in CFA vs. SNI recovery times, so they were grouped here. *** p<0.001; ** p<0.01; *p<0.05.

iDC nerve block has previously been shown to be reversible. This is an important feature for neuromodulation paradigms that have potential for clinical translation. Therefore, how suppressed neuronal activity was recovered post-iDC was explored, as illustrated in FIG. 17. Recordings of net neuronal activity were made for up to 30 minutes following iDC cessation or until a full recovery was qualitatively observed. A suppressed neuron was considered fully recovered once its activity was not significantly different (paired t-test, p<0.05) from the pre-iDC recording. While it has already been shown that iDC did not significantly reduce innocuous activity at a whole-population level, as illustrated in FIGS. 15A and 15B and 16A and 16B, individual tactile- and proprioceptive-evoked neurons occasionally had their activity suppressed (i.e., a statistically significant reduction in their pre- vs. during-iDC evoked activity), enabling analysis of recovery times in these neurons too. A full recovery occurred in 100% of suppressed tactile- and proprioceptive-evoked neurons, 70% of noxious thermal-evoked neurons, and 22.22% of pinch-evoked neurons. No significant differences were found between CFA vs. SNI recovery times (LMER, p>0.05).

The time (min) to full recovery was significantly affected by stimulation type (LMER, p<0.001, n=182). Neurons evoked by the noxious thermal stimulus took the longest to recover on average (EMM=9.96, CI=8.96, 10.97; df=124), followed by pinch-evoked (EMM=3.98; CI=2.32, 5.64; df=125), tactile-evoked (EMM=1.32; CI=0.67, 1.97; df=125), and then proprioceptive-evoked (EMM=1.00; CI=−0.023, 2.02; df=121) neurons. The noxious thermal recovery was significantly longer than all others (all p<0.001). Recovery from pinch was significantly longer than from the proprioceptive (p=0.016) and tactile (p=0.020) stimuli. There was no difference between the tactile and proprioceptive stimulus recovery times (p=0.95).

Finally, background (spontaneous) activity was summed to investigate if it was affected by iDC, as there is a concern that electrical neuromodulation can increase basal neuronal firing. Stimulation type (LMER, p<0.001), but not iDC phase (LMER, p=0.15) had a significant effect. Post-hoc analysis revealed there was increased spontaneous activity in noxious pinch-evoked neurons only (Tukey p=0.046).

In this exemplary implementation, when iDC was delivered to the sciatic nerve of injured rats, evoked nociceptive activity was significantly reduced, while evoked innocuous activity was not. Specifically, iDC suppressed neuronal activity evoked by a sharp pinch and a hot thermal stimulus. These are physiologically meaningful stimuli which represent likely sources of pain for patients in everyday life. iDC's selective suppression is significant because nociceptive information is well-known to be transmitted along primary sensory neurons via small-diameter C- and Aδ fibers. This suggests iDC exerted its effects by selectively suppressing small-diameter fibers while leaving large-diameter (A) fibers unaffected. Selective modulation of small-diameter fibers represents an unprecedented level of neural control, supporting iDC as a promising interventional technology.

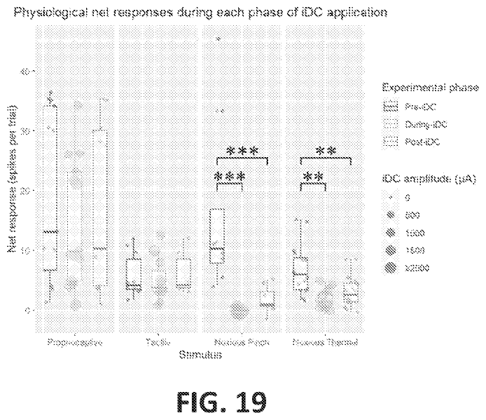

FIG. 19 illustrates a graphical view of an effect of iDC on net neuronal activity in healthy animals. Similarly, to the current study's findings, iDC reduced only the neuronal activity evoked by noxious pinch and thermal stimuli. Innocuous tactile- and proprioceptive-evoked activity was not significantly affected. No recovery from during-iDC to post-iDC was observed. *** p<0.001; ** p<0.01.

Unlike previous iDC studies that report complete nociceptive block, here only partial suppression was observed. For example, iDC reduced noxious thermal-evoked activity by ~55% (28 spikes/trial down to 12.83) at both 500 and 1000 μA, as illustrated in FIGS. 15A and 15B. This was a statistically significant reduction but not to the extent previously reported at comparable amplitudes (~90% block at 800 μA), or other previous findings in healthy animals, as illustrated in FIG. 19. The mechanisms of nerve block with iDC are reasonably well understood. Depolarization of neuronal membrane potentials tonically inactivates VGSCs (cathodic block), or hyperpolarization of neurons holds them below the threshold of VGSC activation (anodic block). Therefore, one possible explanation for these findings is that while iDC blocked the majority of peripheral nociceptive input as expected, any unblocked input was amplified due to pathological central sensitization in the pain models. Chronic pain invokes spinal microglial activation, reduced descending inhibition, and enhanced glutamate release, increasing dorsal horn neuronal excitability. Here, iDC was acting locally on the sciatic nerve, so not affecting these central changes within the time frame studied. Therefore, even small amounts of unblocked primary nociceptive input could explain why dorsal horn activity was not found to be completely silenced. Over longer periods of time, it may be possible that iDC could indirectly reverse central changes by rectifying pathological peripheral input; however, this has not yet been investigated. Chronic iDC studies may reveal more insight into this.

Partial suppression of nociceptive activity may also be explained by specific electrode configuration. The tripolar configuration generates a more complex electric field around the nerve. This has been shown to have a greater stochastic and graded effect on neuronal membrane potentials, aligning with the observations associated with a device according to the present invention. From a clinical perspective, the capacity for a partial or graded block may be superior as it provides finer control over the extent of nociceptive inhibition rather than an ‘all or nothing’ outcome.

iDC was equally effective in two different models of chronic pain. An inflammatory (CFA) and a neuropathic (SNI) model were chosen due to their distinct pathophysiology, to test iDC's utility. Chronic inflammation has an especially profound nociceptive component due to peripheral sensitization following tissue injury. The results reflected this, with CFA (inflammatory) rats displaying significantly greater activity on average across all trials, as illustrated in FIGS. 15A and 15B. Despite this, iDC w as equally effective in both injury models. The results here are limited, because direct statistical comparison to iDC's effects in healthy controls, as illustrated in FIG. 19 has not been done. This will form an important part of the analysis once the datasets are combined for future publication. Nevertheless, the current findings alone suggest iDC is efficacious across various peripheral pain conditions, regardless of etiology. This is clinically advantageous as existing electrical neuromodulation treatments tend to demonstrate efficacy only within very select patient populations, such as those with lower back pain iDC may avoid this pitfall and offer relief for numerous pain conditions.

No differences between the effects of anodic and cathodic iDC were found, however, this was not unexpected. While early DC modelling and isolated nerve conduction studies report lower block thresholds for anodic iDC, more recent in vivo studies with complex physiological parameters show little or no difference. DC neuromodulation outcomes are known to vary with even small changes in electric field geometry. Factors including axon diameter/myelination, axon-electrode distance/contact, electrode array (mono-/bi-/tripolar), and distance from the soma can affect this. Such variables can be difficult to control in vivo, so it is not surprising to see discrepancies compared to in silico modelling predictions. Especially in chronic pain models, where the environment surrounding the nerve is inflamed and may have scar tissue, the DC electric field-nerve interaction is not occurring in ideal conditions. Nevertheless, the fact that polarity had no effect in the study suggests iDC's effects are robust. The finding that iDC does not induce a general increase in background (spontaneous) activity is also promising, as this would defeat the purpose of trying to reduce nociceptive activity. While there was an increase specifically during pinch trials, this could be explained by pinch-sensitive neurons' tendency to “wind-up” in response to repeated stimulation, as in these experiments. Wind-up is a well-known phenomenon that occurs independently of iDC. A follow-up study with more spaced-out pinch trials could confirm iDC is not the cause of this increased activity.

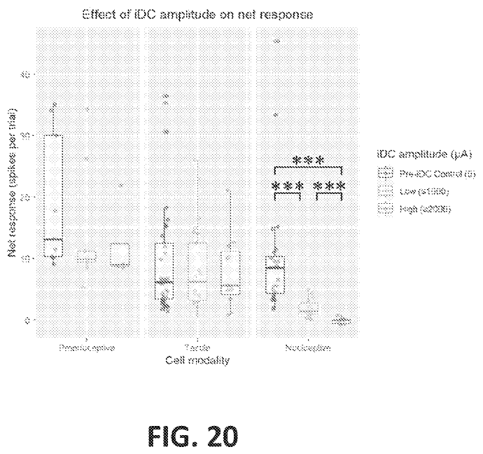

FIG. 20 illustrates a graphical view of an effect of amplitude on net neuronal activity. In contrast to the current study's findings, the amplitude of iDC did significantly affect the extent of nerve block in healthy animals. Future studies examining exact suppression thresholds, likely falling between 50-500 μA, should be conducted. *** p<0.001.

Another critical parameter of electrical neuromodulation is current amplitude. It dictates how much charge is delivered to nervous tissue, which is analogous to the ‘dosage’ of an electrical-based treatment. Two amplitudes of iDC (500 and 1000 μA) were tested and found that the effects of iDC did not significantly differ between them. This was unexpected and does not align with previous studies, which report a strong positive correlation between amplitude and degree of nerve block. This includes a study which found increased iDC block at greater amplitudes, as illustrated in FIG. 20. However, this study tested higher values (1000 μA and 2000 μA) so the outcomes are not directly comparable. Here, improved iDC nerve cuff electrodes with lower impedances and better electrode-nerve contact were used. As discussed above, such factors can influence the effectiveness of DC neuromodulation. Improvements likely caused lower amplitudes to have greater effects. However, this does not explain why a dose-response effect was not observed. It is possible there is a maximal (asymptotic) level of iDC suppression which had already been surpassed at 500 μA. This would explain why the 1000 μA had no significant additional effect. This is what the data suggests, but it remains unclear why other studies using comparable amplitudes did not find this. The use of chronic pain models may offer an explanation. It is possible 500 and 1000 μA was too small of a range to have significantly different effects within the hyperexcited pain systems. Data was collected from only two amplitudes as it enabled recording from a greater number of unique cells within the timeframe of one experiment. As such, the results should not be interpreted to mean increased iDC amplitude has no further effect on nerve suppression and at higher amplitudes (e.g., >2000 μA) greater nociceptive block and partial innocuous fiber block would be observed. This should be confirmed in follow-up studies to examine whether iDC can invoke full nerve block in chronic pain models. Nevertheless, suppression at smaller currents is preferred from a clinical perspective. Lower voltage power sources and higher impedance nerve cuffs can be used. This translates to smaller devices for implantation and lower power consumptions, important considerations for implantable electronics. A dose-response of amplitudes between ~50-500 μA provides a range for the lowest and most effective amplitude in chronic pain conditions.

The final component of iDC's effects is recovery from suppression. First, after iDC was switched off, suppressed nociceptive activity significantly increased towards pre-iDC levels, but not completely. Post-iDC activity remained on average significantly lower than pre-iDC activity, as illustrated in FIGS. 15A and 15B. One possibility is that iDC was simply damaging the nerve. This concern is the main reason recovery time was observed. The implementations were structured so that many subsequent sets of recordings were observed; given iDC is applied homogenously to the whole nerve, if the nerve was being killed, no recordings past the first set could be obtained. Instead, strong responses were evoked for 10-12 hours. Therefore, nerve damage was not the cause of reduced neuronal activity. However, future studies could examine histology of the nerve post-iDC to confirm this.

A more likely possibility is that neurons take longer than 30 minutes to recover from iDC, which was the longest time possible to wait per recovery set. This may be expected, because DC-based neuromodulation block is known to persist following cessation. Given that partial recovery is occurring, a full return to baseline is possible given more time. Neurons could take up to 120-180 minutes to recover. Longer recovery times for single-unit activity than summed population responses may also explain why longer recover times were observed, compared to a rapid 5-minute recovery for population (multi-unit) activity following iDC cessation.

The second recovery finding was that among the sub-population of neurons that did fully recover, nociceptive neurons remained suppressed for significantly longer than innocuous neurons, as illustrated in FIG. 17. This is consistent with previous reports, but the reasons for this difference are not yet understood. Possible hypotheses include differences in gating kinetics of ion channels expressed on specific neuronal sub-populations, or prolonged accumulation of ion species within the extracellular space having long-term effects on driving forces. Explanations become more difficult when pathological changes to ion channel expression due to chronic pain are considered. Downregulation of the K+/Cl− cotransporter 2, for example, is known to disrupt the regulation of K+ and Cl− ion concentrations and in turn membrane excitability. Further investigations into these mechanisms should be pursued, as a greater understanding of recovery times may inform an innovative iDC treatment paradigm. iDC could be delivered at high amplitudes to rapidly suppress all nociceptive activity for a short period of time, then be switched off. The results herein suggest that any affected innocuous signals would recover near-instantaneously while nociceptive neurons would remain suppressed. Periodic cycling of iDC (e.g., short on, long off) may be sufficient to keep slow-recovering nociceptive neurons below a certain level of activity, yielding a long-duration analgesic effect from short-duration iDC. This is of course speculative but highlights the potential of future investigations in this area.

The aims of this study depend on the successful establishment of chronic pain models. The animal models used are widely recognized to induce allodynia and hyperalgesia, two chronic pain symptoms shared between rodents and humans. As expected, both CFA and SNI rats developed intense mechanical hypersensitivity (allodynia) following injury. Qualitative increases in known pain behaviors such as licking, guarding, and lifting of the injured hindpaw were also observed. Thresholds did not change from day 3 to day 4 (CFA rats) or 7 (SNI rats), suggesting mechanical allodynia was robust.

Increased thermal pain sensitivity also developed in CFA rats but not in SNI rats. This aligns with literature showing that heat hypersensitivity does not develop in the spared tibial SNI variation that was used. The reasons for this are unclear but may be due to the specific fiber-type composition of the rat tibial nerve. Regardless, the tibial variation was used as electrophysiological recordings were too difficult to obtain using the original model, which only leaves the slim sural dermatome innervated. Chronic pain-like changes were established in all rats by the time of the electrophysiology experiments. This conclusion is reinforced by a recent electrophysiology study showing significant increases in the excitability and firing of dorsal horn neurons in a similar animal model.

Recent preclinical pain research has reported strong sexual dimorphism, highlighting more pronounced neuropathic pain in females. Across all von Frey trials, females displayed significantly lower withdrawal thresholds; however, they did not develop stronger allodynia as other studies have reported. In contrast, across all Hargreaves trials, males had lower withdrawal latencies. No significant sex effects were found in the electrophysiology experiments. Combined behavioral and electrophysiological data suggests sex differences may be mediated by central mechanisms (e.g., cortical changes, limbic differences, etc.) rather than in the periphery. This could be investigated via cortical electrophysiology pain studies, to add to the growing body of literature highlighting sexual dimorphism in pain and its importance in preclinical and clinical research.

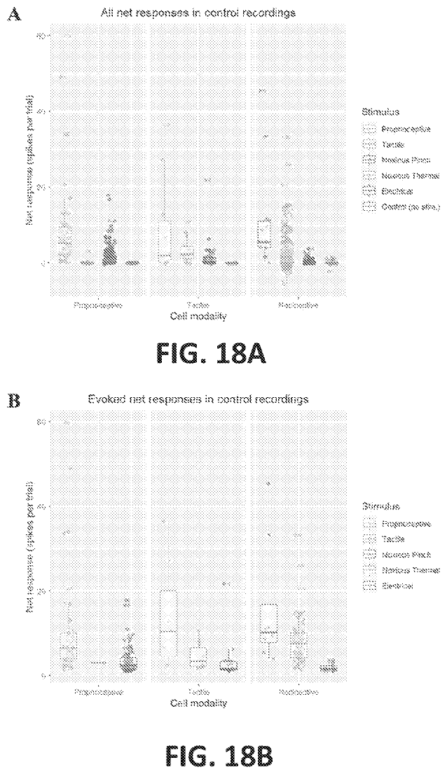

FIGS. 18A and 18B illustrate graphical views of net activity in control recordings by neuron modality (type) in healthy animals. FIG. 18A illustrates net activity from all control recordings made, and FIG. 18B illustrates net activity from only those neurons whose responses were evoked according to criteria.

A critical assumption of this study is that increased neuronal activity evoked by nociceptive stimuli corresponds to an increase in the firing of nociceptive neurons. This relies on having a robust neuron classification system. As described in the methods, a neuron whose spike template was derived from a recording of noxious pinch-evoked activity, and which responded most to the pinch stimulus, was a “noxious pinch-dominant” neuron, for example. This method was previously used with success, finding that nociceptive neurons were exclusively activated by nociceptive stimuli, as illustrated in FIGS. 18A and 18B. However, classifications using this system become less straightforward in chronic pain models where phenomena like allodynia represent tactile neurons becoming nociceptive. Inflammatory pain conditions especially have been shown to induce phenotypic switching, whereby a subpopulation of large-diameter AP neurons adopt a C-fiber-like phenotype, expressing substance P and actively contributing to spinal hyperexcitability. To mitigate the concern of misclassification, the analysis was narrowed by removing nociceptive neurons which also responded to innocuous stimuli. Although this reduced the range of neurons examined, it allowed for confidence that a suppression of pinch- or thermal-evoked activity was a suppression of nociceptive neurons. Future studies could utilize additional classification measures, such as action potential velocity as determined by response latency, to aid in neuron classification. This is possible because noxious information travels in small-diameter, unmyelinated fibers significantly slower than innocuous information does in large-diameter, myelinated fibers.

Evoked responses were qualitatively observed to degrade over time. Activity during-iDC was therefore always compared to the most recent non-iDC recording (<5 min difference). This mitigated the likelihood that a timing effect confounded iDC's suppressive effects, but this remains a possibility. Additionally, DC-based neuromodulation is known to change spike shape. It is possible that the spike-sorting could report a “block” if the action potential shape changed enough that it was not recognized and matched to its own baseline template. This was addressed by manually inspecting random samples of an “unmatched” bin created by the spike-sorting that displayed all non-matched spikes. This effect was not observed in any inspected samples; however, inspections could not be done for every set.

It is clear from existing literature that the effects of DC-based neuromodulation on nerve membrane potentials are highly complex. In a study using an ultra-low frequency waveform like iDC, block of large-diameter (Aβ) fibers at amplitudes where small-diameter (Aδ/C) fibers were only partially suppressed was reported. However, the difference in block thresholds of Aβ and C-fibers reported was significantly less than that observed in high-frequency AC block studies, suggesting there may be a finer balance between what is preferentially suppressed when low-frequency DC-like waveforms are used. Another study showed the same effect in the opposite direction: total block of small-diameter fibers was accompanied by partial suppression of large-diameter fibers. Together, these indicate that complex low-frequency waveforms can elicit fiber-specific nerve blocks to varying degrees. The exact ratio or preference of block is malleable and dependent on the exact waveform parameters used. Here, it is shown that tripolar delivery of iDC likely results in a preferential suppression of small-diameter fibers, and this is maintained in hyperexcited chronic pain environments. With greater understanding of these nuanced mechanistic differences, there is potential for powerful control of neuronal membrane potentials using waveforms like iDC.

iDC can significantly reduce nociceptive neuronal activity, suggesting a preferential suppression of small-diameter (Aδ/C) fibers in the periphery. The data on iDC suggests potential for clinical use in chronic pain. The immediate next step is to combine electrophysiology data with the healthy control data collected previously, as illustrated in FIGS. 18A, 18B, 19, and 20. This will reveal how iDC's effects differ between healthy and injured animals. A qualitative comparison of results, as illustrated in FIGS. 15A, 15B, and FIG. 19, indicates iDC may suppress injured animals' neuronal activity to healthy animals' baseline levels. Should this be the case, iDC could present itself as a treatment capable of restoring normal pain sensation, an outcome which would be even more preferrable than total removal of pain sensations.

iDC may also play an important role in treating currently intractable pain conditions. Osteo- and rheumatoid arthritis, mononeuropathies like carpal tunnel syndrome, and localized cases of chronic diabetic peripheral neuropathy or chemotherapy-induced peripheral neuropathy may be relieved by iDC. These are severe conditions requiring an urgent solution. Important precedents supporting clinical iDC studies have recently been set with the testing of peripheral neuromodulation devices in humans.

Finally, outside the realm of pain, selective modulation of small-diameter fibers has several important applications. In the area of prosthetics, for example, recruitment in motor control naturally occurs in the order of small- to large-diameter. This is relevant for greater grading of muscle force to improve neural prosthetics for amputees and something which iDC may be able to contribute.

iDC can be used to suppress pain-related neuronal activity, without impairing activity related to the sensations of touch or movement. These results strongly suggest that iDC is worthy of further investigation as a potential treatment for chronic pain conditions, especially those affecting the periphery. Independent of its use in this way, the results provide a strong motivation and direction for further exploration of iDC as a versatile neuromodulation paradigm. The possibilities across science and medicine are vast and exciting. The time to explore these is now.

In the exemplary implementation described. iDC was delivered to the sciatic nerve via a silicone nerve cuff electrode, 2 mL each of parts A and B of Rebound-40 silicone rubber (Smooth-On, PA, USA) were then mixed and poured over the slide, which was immediately spin-coated at 600 rpm for 10 seconds to obtain an even silicone coating. While this cured, a custom-designed, paper-based, three-channel (i.e., tripolar) template was cut out using a Cricut machine (UT, USA). The paper template was sprayed with two coats of Ease Release 200 non-stick agent (Mann Release Technologies, PA, USA) before being gently pressed into the cured silicone layer on the glass slide. Another layer of silicone was poured over the paper template (1.5 mL each of parts A and B) and again spin-coated at 600 rpm for 10 seconds. Once cured, a final layer (1 mL each of parts A and B) was poured and spin-coated at the same settings. Once this final layer had cured, a thin incision was made to extract the paper template, leaving three hollow channels within the silicone cuff. A plastic tube was then attached to the top-end of each channel and a 3% w/v agar electrolytic solution was heated and syringed into the tubes to fill the three channels. The agar solidifies upon cooling, leaving solid agar-filled channels within the cuff. The tubes/channels were then connected to an A-M Systems 4100 DC power supply. DC was thus delivered to the sciatic nerve via the electrolytic agar channels as a stream of ions i.e., as ionic direct current. The completed silicone cuff is shown in FIG. 7A. The fabrication and use of this cuff is significant as it represents a clinically feasible way to deliver iDC via an implantable device. There is growing interest in biomedical engineering research for such implantable nerve cuffs for pain.

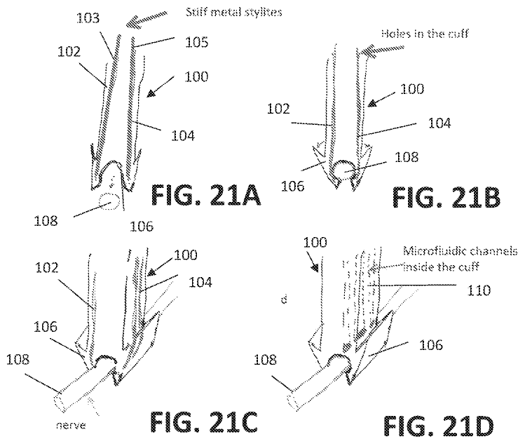

FIGS. 21A-21D illustrate a cuff design and method of insertion, according to an embodiment of the present invention. FIGS. 21A-21D illustrate a cuff design 100 that allows for the cuff to be positioned around the nerve 108 with no flap to be draped around the nerve. The design of the cuff 100 shown in FIGS. 21A-21D allows for a minimally invasive implantation through a needle. FIG. 21A illustrates that the cuff 100 contains channels 102 and 104 that allow stiff stylets 103 and 105 to be placed in them to straighten the tip 106 of the cuff 100. The cuff 100 is shown approaching the nerve 108 from the side-view. FIG. 21B illustrates a side view of the cuff 100 with the tip 106 positioned around the nerve 108 once the stylets 103 and 105 are removed from channels 102 and 104. The lines indicate the channels 102 and 104 naturally angled at the tip 106. The cuff 100 can be formed from silicone in some embodiments. Alternately, the cuff 100 can be formed from any other biocompatible material known to or conceivable by one of skill in the art. The cuff 100 is manufactured so that the tip 106 is angled as to encase the nerve 108 and the channels 102 and 104 naturally follow the curvature of the structure of the tip 106. FIG. 21C illustrates a perspective view of the nerve 108 and the cuff 100 positioned around it. FIG. 21D illustrates a perspective view of an internal trajectory of the microfluidic channels 110 and the contact between the microfluidic channels and the nerve 108 at the top of the structure.

In some embodiments, function of the present invention can be carried out in conjunction with a computer, processor, non-transitory computer readable medium, or alternately a computing device or non-transitory computer readable medium incorporated into the medical device associated with the present invention.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method and operation of the present invention.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

1. A device comprising:
a cuff configured to contact a peripheral nerve;
microfluidic channels, wherein each of the microfluidic channels defines an inner space, and wherein each of the microfluidic channels are configured to conduct energy such as ionic direct current to the peripheral nerve to selectively suppress a spiking activity of pain transmitting nerve fibers.

2. The device of claim 1 further comprising a conduit of energy.

3. The device of claim 1 wherein the cuff is formed from a biocompatible material such as silicone.

4. The device of claim 1 wherein the cuff is configured to maintain contact with a surface of the peripheral nerve.

5. The device of claim 4 wherein the cuff is coupled to the microfluidic channels, and the cuff contacts the peripheral nerve.

6. The device of claim 1 wherein each of the microfluidic channels is filled with a conductive media such as a gel.

7. The device of claim 1 wherein each of the microfluidic channels is connected to a direct current source.

8. The device of claim 7 wherein the microfluidic channels have a tripolar configuration.

9. The device of claim 8 wherein the tripolar configuration includes a central channel flanked by two return channels.

10. The device of claim 7 wherein delivery of ionic direct current occurs in three phases: a 10 s ramp on, 90 s constant ionic direct current delivery, and a 10 s ramp off.

11. A device comprising:
three channels, wherein each of the three channels defines an inner space, and wherein the three channels are configured to conduct energy such as ionic direct current to a target.

12. The device of claim 11 further comprising a cuff for delivering energy to the target.

13. The device 11 of claim further comprising a conduit of energy.

14. The device of claim 12 wherein the cuff is formed from a biocompatible material such as silicone.

15. The device of claim 12 wherein the cuff is configured to contact a surface of a peripheral nerve.

16. The device of claim 11 wherein each of the three channels is filled with a conductive media such as a gel.

17. The device of claim 16 wherein each of the three channels is connected to a direct current source.

18. The device of claim 17 wherein the three channels have a tripolar configuration.

19. The device of claim 18 wherein the tripolar configuration includes a central channel flanked by two return channels.

20. The device of claim 17 wherein delivery of ionic direct current occurs in three phases: a 10 s ramp on, 90 s constant ionic direct current delivery, and a 10 s ramp off.
